(19) United States
(12) Patent Application Publication
(10) Pub. No.: US 2008/0119835 A1
(43) Pub. Date: May 22, 2008

(54) DEVICE FOR USE DURING SURGICAL PROCEDURES

(75) Inventors: William Richard Salter, Garland, TX (US); James Ryan Mujwid, Crystal, MN (US)

Correspondence Address: AKIN GUMP STRAUSS HAUER & FELD, LLP, P O BOX 688, DALLAS, TX, 75313-0688, US

(73) Assignee: Dr. William Richard Salter, Garland, TX (US)

(21) Appl. No.: 11/944,080

(22) Filed: Nov. 21, 2007

Related U.S. Application Data
(60) Provisional application No. 60/866,630, filed on Nov. 21, 2006.

(51) Int. Cl. A61B 18/02; B23Q 3/02; F25D 3/00

(52) U.S. Cl. 606/20; 269/309; 62/293

(57) ABSTRACT

One embodiment of the present invention includes a device for use in a surgical procedure. The device includes a first curved portion for holding a top portion of a surgical probe; a second curved portion for holding a handle of the surgical probe, wherein the first curved portion and the second curved portion are adjoined through a frame; and an attachment at a bottom of the device, wherein the attachment can attach the device to a tripod.

### CROSS-REFERENCE TO RELATED APPLICATIONS

This application claims priority to and benefit of U.S. Provisional Application Ser. No. 60/866,630, filed on Nov. 21, 2006, entitled: ACCESSORY OR DEVICE FOR USE DURING CRYOABLATION TREATMENTS, by inventor, William Richard Salter [Attorney Docket No. 685717-0002].

### STATEMENT REGARDING FEDERALLY SPONSORED RESEARCH OR DEVELOPMENT

Not Applicable.

### REFERENCE TO A “MICROFICHE APPENDIX”

Not Applicable.

### TECHNICAL FIELD OF THE INVENTION

The present invention relates generally to a device for use during surgical procedures, and specifically, during cryosurgical uterine ablation.

### BACKGROUND OF THE INVENTION

Cryosurgical probes are used to freeze and therefore destroy human tissue. These probes are commonly used to freeze tissue in a woman's uterus. A physician using these probes often has to hold the probe in place for large amounts of time. Thus, the physician's hands and/or shoulders may experience fatigue from time to time.

Various devices and methods related to cryosurgical probes have been developed and disclosed in U.S. Pat. Nos. 6,530,234; 6,237,355; 6,475,212; 6,193,644; 6,471,694; 6,182,666; 6,451,012; 6,035,657; 6,270,494; 5,910,104; 6,241,722; and 5,758,505 which are each incorporated by reference herein.

Therefore, a need exists for a device to assist a physician in holding a probe in place for extended periods of time during surgical procedures.

### BRIEF SUMMARY OF THE INVENTION

An embodiment of the present invention includes a device to assist in maintaining and supporting a cryosurgical probe in the proper position and at the proper height. In addition, the device helps keep the probe stable during treatment. The device helps keep the probe in one location to give the physician an opportunity to move around and to avoid holder fatigue.

The following description and drawings set forth in detail a number of illustrative embodiments of the invention. These embodiments are indicative of but a few of the various ways in which the present invention may be utilized.

### BRIEF DESCRIPTION OF THE DRAWINGS

For a more complete understanding of the present disclosure and its advantages, reference is now made to the following description taken in conjunction with the accompanying drawings, in which like reference numerals represent like parts:

FIG. 1 is a perspective view of an embodiment of the present invention;

FIG. 2 is a side view of one possible surgical device that can be used with an embodiment of the present invention;

FIG. 3 is a top view of an embodiment of the present invention;

FIG. 4 is a rear view of an embodiment of the present invention;

FIG. 5 is a front view of an embodiment of the present invention;

FIG. 6 is a bottom view of an embodiment of the present invention;

FIG. 7 is a detailed view of FIG. 6;

FIG. 8 is a cross section of FIG. 6;

FIG. 9 is a cross section of FIG. 8; and

FIG. 10 is another cross section of FIG. 8.

### DETAILED DESCRIPTION OF THE INVENTION

The following discussion is presented to enable a person skilled in the art to make and use the invention. The general principles described herein may be applied to embodiments and applications other than those detailed below without departing from the spirit and scope of the present invention as defined herein. The present invention is not intended to be limited to the embodiments shown but is to be accorded the widest scope consistent with the principles and features disclosed herein.

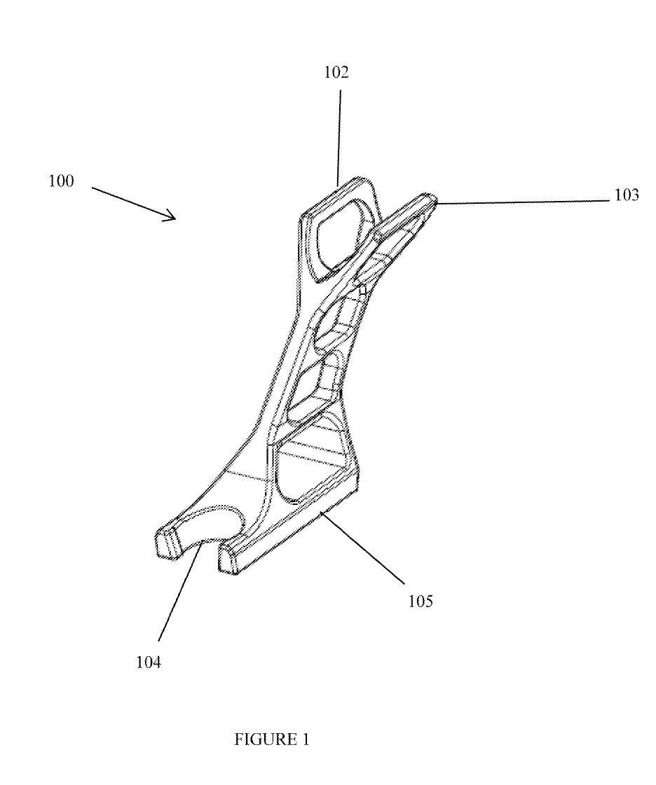

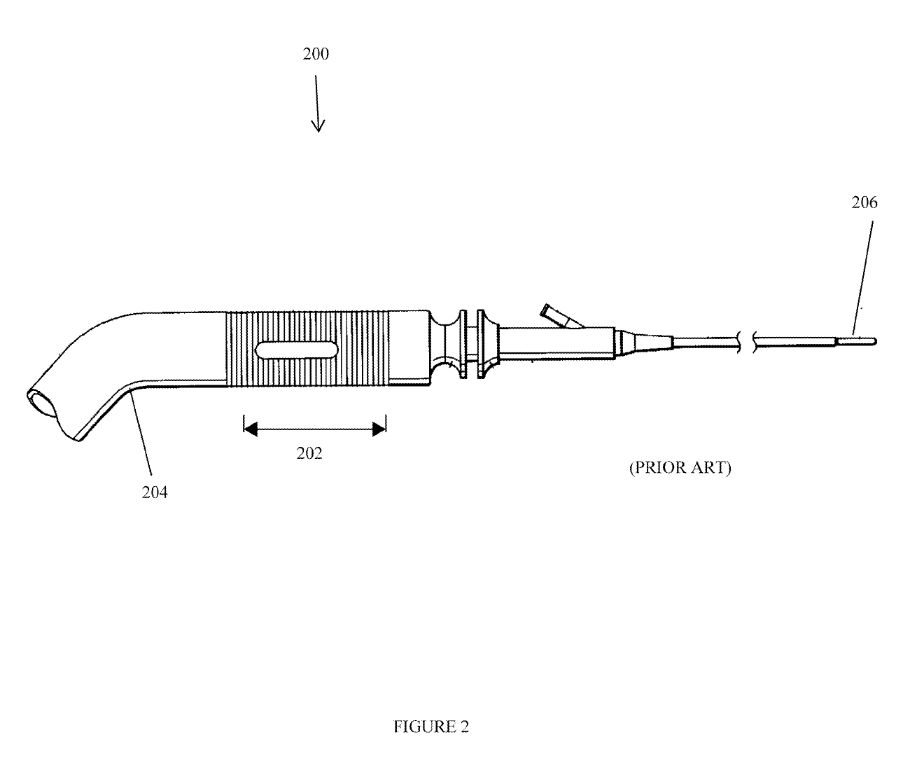

FIG. 1 illustrates a perspective view of an embodiment of the present invention. The holding device 100 includes a frame-like structure with a top curved area 102, in the front 103 of the device, that a surgical device (as shown, but not limited to, in FIG. 2) can rest on. In addition, the holding device 100 includes a bottom curved area 104, in the back 105 of the device, where a handle (not shown) of the surgical device can rest on. However, in this embodiment, the holding device 100 is actually attached to a tripod (not shown) that be adjusted in height.

FIG. 2 is a side view of one possible surgical device that can be used with an embodiment of the present invention. In this embodiment, a cryosurgical probe 200 is shown. In this example, the cryosurgical probe 200 is a closed loop Joule-Thomson cryosurgical probe 200. The cryosurgical probe 200 can be used with the holding device 100 for freezing and thereby destroying biological tissues. It is contemplated that the cryosurgical probe 200 can be used with the holding device 100 so that a physician can perform a uterine ablation by freezing areas of tissue within a women's uterus. The cryosurgical probe 200 includes a portion 202 of the probe that would rest on the top curved area 102 (shown in FIG. 1) of the holding device 100. In addition, the handle 204 of the probe 200 would rest on the bottom curved area 104 (shown in FIG. 1) of the holding device 100.

One method of use includes attaching the holding device 100 to a tripod (not shown) and then placing the probe 200 on the holding device 100. The height of the holding device 100 may be adjusted at this point to allow the physician to position the probe 200 for entry into a woman's uterus. After the physician is satisfied with the height of the holding device 100 and probe 200, the physician holds the probe handle 204 until the probe 200 is frozen in place, usually about 1 minute and 30 seconds. After this time frame, there usually is not a need for a tightening or fastening the holding device 100. Once the probe tip 206 is frozen in place, the probe 200 usually does not move in or out of the uterus. The holding device 100 can tip up or down but usually doesn't need to because the height was adjusted earlier. The only time the probe handle 204 is removed from the holding device 100 is to reposition or remove the probe 200. Otherwise, the probe handle 204 sits in the holding device 100 to make it easier for the physician to keep a sonogram transducer stable on the abdomen as the physician watches the tip of the probe 200 and the ice ball formation. This method provides the physician relief from shoulder and arm fatigue.

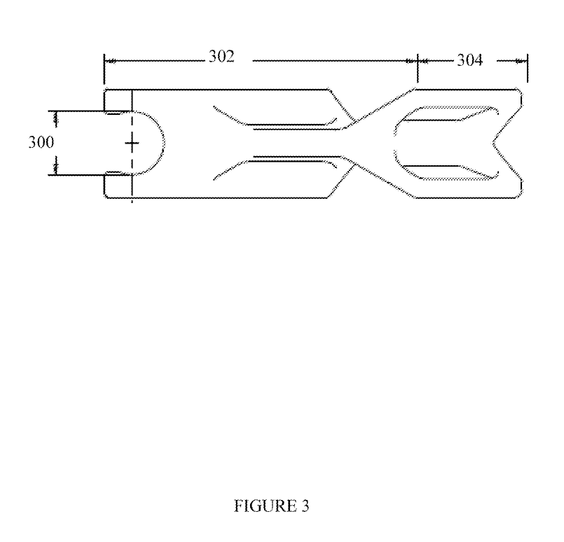

Now, details of the construction of the holding device 100 are described in relation to FIGS. 3-10. Specifically, FIG. 3 is a top view of an embodiment of the present invention. In this embodiment, the bottom curved area 104 includes a diameter 300 of about 1.1 inches. In addition, the distance 302 from an end of the bottom curved area 104 to a top curved area 102 is about 5.7 inches. Moreover, the distance 304 from an end of the top curved area 102 to the other end is about 2 inches.

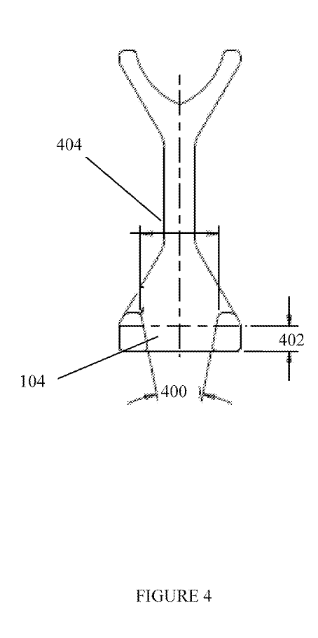

FIG. 4 is a rear view of an embodiment of the present invention. The bottom curved area 104 has an angle to grip the handle 204 of the probe 200. In this embodiment, the angle 400 from the top of the bottom curved area 104 to the bottom of the bottom curved area 104 is about 22 degrees. In addition, the diameter 404 of the bottom curved area 104 is about 1.28 inches. Moreover, the distance 402 from the bottom of the bottom curved area 104 to just where the sides start to curve is about 0.4 inches.

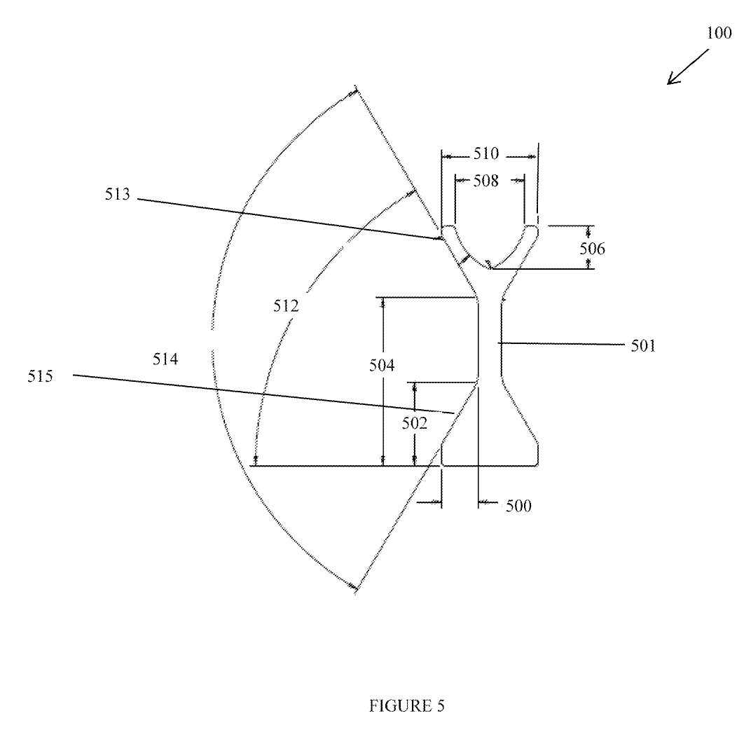

FIG. 5 is a front view of an embodiment of the present invention. In this embodiment, a distance 500 between the straight center portion 501 of the holding device 100 and one end of holding device 100 is about 0.75 inches. Moreover, the height 502 of the angled portion to the straight portion 501 is about 1.75 inches. In addition, the height 504 of that portion is about 1.75 inches. Moreover, the distance 504 between the bottom of the holding device 100 and the top of the straight center portion 501 is about 3.5 inches. Further, the distance 506 between the top of the holding device 100 and the center of the top curved area 102 is about 0.9 inches. Additionally, a distance 508 between the inner portion of the top curved portion 102 is about 1.5 inches while the distance 510 between one end of the top to the other end of the top is about 2 inches. Furthermore, the angle 512 of the top angled portion 513 to the horizontal is about 60 degrees while the angle 514 between the top angled portion 513 and the bottom angled portion 515 is about 120 degrees.

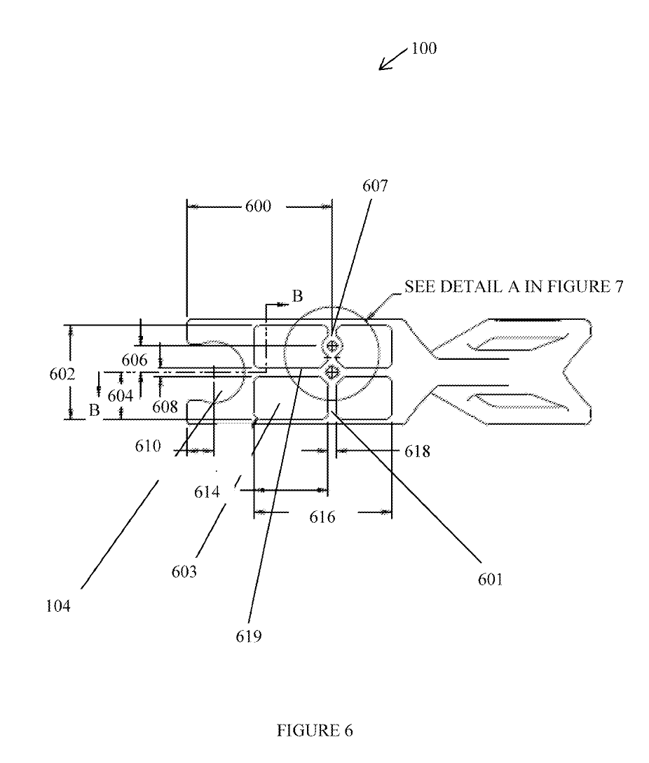

FIG. 6 is a bottom view of an embodiment of the present invention. In this embodiment, a distance 600 between the center of the vertical bar 601 and one end of the holding device 100 is about 2.7 inches. In addition, a distance 602 between the ends of the cutout portions 603 within the holding device 100 is about 1.8 inches while the distance 604 between the vertical center of the device and an end of the cutout portions is about 0.9 inches. Moreover, the distance 606 between the one end of the device and a center of a first attached bolt 607 is about 0.5 inches. Further, the width 608 of the vertical bar 601 is about 0.15 inches. Additionally, the distance 610 between the center of the bottom curved portion 104 and the end of the device is about 0.5 inches. Furthermore, the width 614 of a first set of cutout portions 603 is about 1.4 inches while the width 616 of both sets of cutout portions is about 2.6 inches. In addition, the width 618 of the horizontal bar 619 is about 0.15 inches.

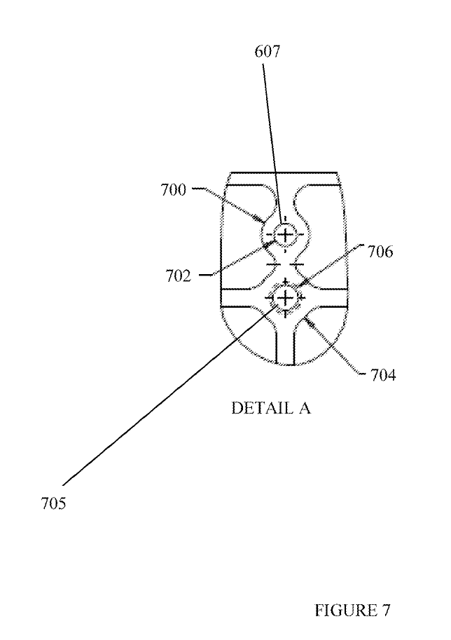

FIG. 7 is a detailed view of FIG. 6. In this embodiment, the circumference 700 of the portion around the first attached bolt 607 is about 0.38 inches while the circumference 702 of the first attached bolt 607 is about 0.18 inches. Moreover, the circumference 704 of the portion around a second attached bolt 705 is about 0.44 inches while the circumference 706 of a second attachment bolt 705 is about 0.38 inches.

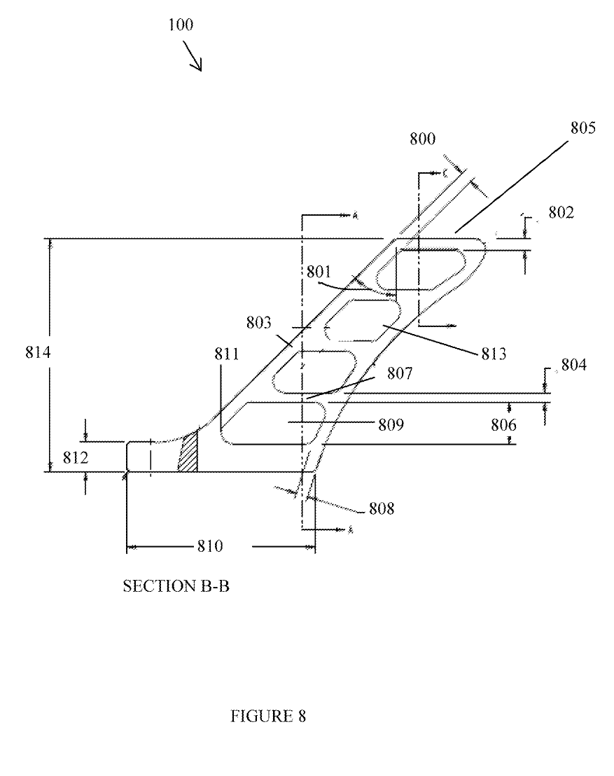

FIG. 8 depicts cross section B-B of FIG. 6. In this embodiment, the width 800 of a first diagonal bar 803 is about 0.25 inches while an angle 801 is about 45 degrees. In addition, a width 802 of the top vertical bar 805 is about 0.25 while a width 804 of a center bar 807 is about 0.20 inches. However, a width 806 of the cutout portions 809 is about 0.9 inches. Moreover, the width 808 a second diagonal bar 813 is about 0.25 inches. Further, the length 810 of the bottom section 811 is about 4 inches while the height 814 of the holding device 100 is about 5 inches. However, the thickness 812 of the bottom 811 is about 0.6 inches.

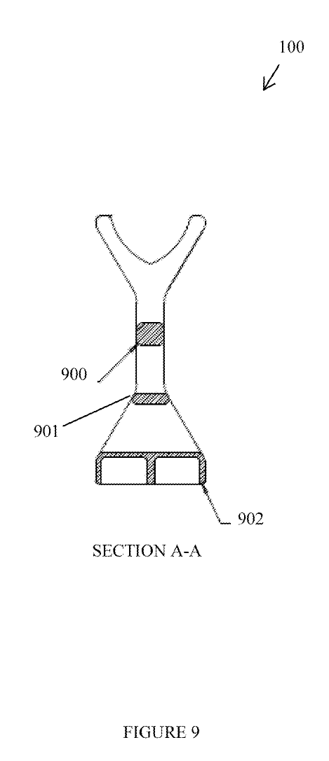

FIG. 9 depicts cross section A-A of FIG. 8. In this figure, center bars 900 and 901 are shown in cross section. In addition, the figure also shows how the holding device 100 is formed in area 902 of the cutout portions shown in FIG. 6.

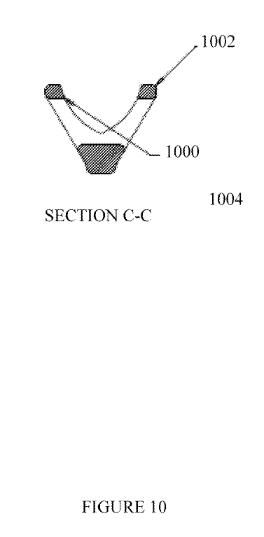

FIG. 10 depicts cross section C-C of FIG. 8. In this figure, top bars 1000 and 1002 and center bar 1004 are shown in cross section.

The previous description of the disclosed embodiments is provided to enable those skilled in the art to make or use the present invention. Various modifications to these embodiments will be readily apparent to those skilled in the art and generic principles defined herein may be applied to other embodiments without departing from the spirit or scope of the invention. For example, the hybrid server and storage array may be implemented in a small box that is artistic and may be mountable on a wall. Thus, the present invention is not intended to be limited to the embodiments shown herein but is to be accorded the widest scope consistent with the principles and novel features disclosed herein.

What is claimed is:

1. A device for use in a surgical procedure, the device comprising:
a first curved portion for holding a top portion of a surgical probe;
a second curved portion for holding a handle of the surgical probe, wherein the first curved portion and the second curved portion are adjoined through a frame; and
an attachment at a bottom of the device, wherein the attachment permits engagement of the device to a tripod.

2. The device of claim 1 wherein the surgical procedure includes cryosurgical ablation.

3. The device of claim 1 wherein the surgical probe includes a cryosurgical probe used for cryosurgical uterine ablation.
